(19) United States
(12) Patent Application Publication
(10) Pub. No.: US 2025/0275950 A1
(43) Pub. Date: Sep. 4, 2025

(54) RIFAXIMIN AND N-ACETYL CYSTEINE FORMULATIONS

(71) Applicant: Cedars-Sinai Medical Center, Los Angeles, CA (US)

(72) Inventors: Mark Pimentel, Los Angeles, CA (US); Gabriela Leite, Porter Ranch, CA (US); Ruchi Mathur, Los Angeles, CA (US); Ali Rezaie, Beverly Hills, CA (US)

(73) Assignee: Cedars-Sinai Medical Center, Los Angeles, CA (US)

(21) Appl. No.: 18/862,311

(22) PCT Filed: May 19, 2023
(86) PCT No.: PCT/US2023/067221

§ 371 Date: Nov. 1, 2024

Related U.S. Application Data
(60) Provisional application No. 63/343,860, filed on May 19, 2022.
(60) Provisional application No. 63/343,862, filed on May 19, 2022.

(51) Int. Cl. A61K 31/437; A61K 31/198; A61P 1/00; A61P 1/12

(52) U.S. Cl. CPC A61K 31/437; A61K 31/198; A61P 1/00; A61P 1/12

(57) ABSTRACT

Described herein are rifaximin and N-acetylcysteine (NAC) formulations. Method of using the formulations to treat various gastrointestinal ailments, including IBS-D and SIBO are also provided.

### CROSS-REFERENCE TO RELATED APPLICATIONS

This application includes claims of priority under 35 U.S.C. § 119(e) to U.S. provisional patent application No. 63/343,860, filed May 19, 2022, and No. 63/343,862, filed May 19, 2022, the entirety of both is hereby incorporated by reference.

### FIELD OF INVENTION

This invention relates to formulations of rifaximin and N-acetylcysteine and their uses in gastrointestinal purposes.

### BACKGROUND

All publications herein are incorporated by reference to the same extent as if each individual publication or patent application was specifically and individually indicated to be incorporated by reference. The following description includes information that may be useful in understanding the invention described herein. It is not an admission that any of the information provided herein is prior art or relevant to the presently claimed invention, or that any publication specifically or implicitly referenced is prior art.

Small intestinal bacterial overgrowth (SIBO) occurs when there is an abnormal increase in the overall bacterial population in the small intestine—particularly types of bacteria not commonly found in that part of the digestive tract.

Irritable Bowel Syndrome (IBS) is the most common gastrointestinal disorder, with an estimated prevalence in the US of 10% to 15%. IBS is characterized by symptoms which include abdominal pain, bloating, and chronic changes in bowel function that may present as diarrhea-predominant (D-IBS), constipation-predominant (C-IBS) or alternate between the two (M-IBS).

Bacterial alterations of the gastrointestinal tract have been linked to the development of symptoms in IBS. Antibacterial agents often cannot penetrate the mucus layer adequately to control infections, but at the same time, the mucus layer provides an important protective role.

The inventors have found and described various compositions for and methods of treating IBS in International Publication No. WO 2020/191076, which is herein incorporated by reference as though fully set forth. However, there remains a need in the art to provide additional formulations and methods of treating these conditions.

### SUMMARY OF THE INVENTION

The following embodiments and aspects thereof are described and illustrated in conjunction with compositions and methods which are meant to be exemplary and illustrative, not limiting in scope.

Various embodiments of the invention provide for a rifaximin and N-acetylcysteine (NAC) formulation, comprising: a shell; a first solid composition comprising an amount of rifaximin within the shell; and a plurality of a coated second solid composition comprising an amount of NAC within the shell.

Various embodiments of the invention provide for a rifaximin and N-acetylcysteine (NAC) formulation, comprising: a solid core composition comprising an amount of rifaximin and an amount of NAC; and a coating around the solid core composition.

In various embodiments, the amount of rifaximin can be about 66 mg. In various embodiments, the amount of rifaximin can be about 62-70 mg. In various embodiments, the amount of rifaximin can be about 59-73 mg. In various embodiments, the amount of rifaximin can be about 132 mg. In various embodiments, the amount of rifaximin can be about 125-139 mg. In various embodiments, the amount of rifaximin can be about 118-146 mg.

In various embodiments, the amount of NAC can be about 560 mg. In various embodiments, the amount of NAC can be about 532-588 mg. In various embodiments, the amount of NAC can be about 504-616 mg. In various embodiments, the amount of NAC can be about 450-560 mg. In various embodiments, the amount of NAC can be about 427-588 mg. In various embodiments, the amount of NAC can be about 405-616 mg.

In various embodiments of the formulation having the first solid composition and the second solid composition, the first solid composition can further comprise one or more excipients. In various embodiments of the formulation having the first solid composition and the second solid composition, the second solid composition can further comprise one or more excipients. In various embodiments, the first solid composition can be in a tablet form.

In various embodiments of the formulation having the first solid composition and the second solid composition, the second solid composition can be in form of a mini-tablet, crystal, a pellet, or a bead.

In various embodiments of the formulation having the first solid composition and the second solid composition, the coating of the second solid composition can comprise at least one polymer. In various embodiments of the formulation having the first solid composition and the second solid composition, the coating of the second solid composition can comprise at least one polymer that is insoluble at a pH lower than 5. In various embodiments of the formulation having the first solid composition and the second solid composition, the coating of the second solid composition can comprise at least one polymer that is insoluble at a pH lower than 3.5. In various embodiments of the formulation having the first solid composition and the second solid composition, the coating of the second solid composition can comprise at least one polymer that is insoluble at a pH between 1.5-3.5.

In various embodiments of the formulation having the first solid composition and the second solid composition, the shell does not comprise gelatin. In various embodiments of the formulation having the first solid composition and the second solid composition, the shell can comprise hypromellose.

In various embodiments of the formulation having the first solid composition and the second solid composition, first solid composition comprising the rifaximin can be substantially undissolved after 1 hour in a Type 2 dissolution bath containing about 0.1N hydrochloric acid.

In various embodiments of the formulation having the first solid composition and the second solid composition, the first solid composition comprising the rifaximin substantially can disperse in fed state simulated intestinal fluid.

In various embodiments of the formulation having the solid core composition, the solid core composition comprises a single layer that is uniform in composition or essentially uniform in composition.

In various embodiments of the formulation having the first solid composition and the second solid composition, first solid composition comprising the rifaximin can be substantially undissolved after 1 hour in a Type 2 dissolution bath containing about 0.1N hydrochloric acid.

In various embodiments of the formulation having the first solid composition and the second solid composition, wherein the first solid composition comprising the rifaximin is a tablet comprising rifaximin and one or more excipients; wherein the plurality of coated second solid composition comprising NAC is a plurality of a mini-tablet comprising NAC and one or more excipients; and wherein the coating comprises at least one polymer that is substantially insoluble at a pH lower than 3.5.

In various embodiments of the formulation having the solid core composition, the NAC in the solid core composition increases the dissolution of rifaximin relative to a solid core composition without NAC.

In various embodiments of the formulation having the solid core composition, the coating does not comprise gelatin. In various embodiments of the formulation having the solid core composition, the coating can comprise hypromellose.

Various embodiments provide for a method of treating irritable bowel syndrome (IBS), comprising: administering one or more doses of a formulation of the invention as described herein, to a subject in need thereof.

In various embodiments, the IBS can be diarrhea predominant IBS (IBS-D). In various embodiments, the IBS can be hydrogen sulfide (H₂S) positive IBS.

Various embodiments provide for a method of treating small intestinal bacterial overgrowth (SIBO), comprising: administering one or more doses of a formulation of the invention as described herein, to a subject in need thereof.

Various embodiments provide for a method of decreasing Escherichia coli (E. Coli) load, Klebsiella load, and total bacterial load in the small bowel, comprising: administering one or more doses of a formulation of the invention as described herein, to a subject in need thereof.

In various embodiments, the decrease in the E. Coli load, Klebsiella load, and the total bacterial load can occur in the ileum. For example, the formulation is configured to substantially release the rifaximin and NAC in the ileum. In various embodiments, the decrease in the E. Coli load, Klebsiella load, and the total bacterial load can occur in the duodenum. For example, the formulation is configured to substantially release the rifaximin and NAC in the duodenum.

Various embodiments provide for a method of improving stool consistency, reducing systemic inflammation, or both, comprising: administering one or more doses of a formulation of the invention as described herein, to a subject in need thereof.

In various embodiments, administering one or more doses of the formulation can comprise administering three dosages per day to the subject in need thereof. In various embodiments, administering one or more doses of the formulation can comprise administering three dosages per day for about 7 to 10 days to the subject in need thereof. In various embodiments, administering can be via oral administration.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows human and rat conversions derived from in vitro dose discovery experiments examining cidal activity of rifaximin, NAC and both on E. coli and Klebsiella.

FIG. 2 shows the in vivo study plan of rifaximin and NAC in a rat model.

FIGS. 3A and 3B show that therapy with rifaximin+NAC decreases Escherichia coli load and total bacterial load in the small bowel. Escherichia coli had a direct association with % of H₂O in stool. (Spearman r=0.141, P=0.037)

FIG. 4 shows that therapy with rifaximin+NAC improves stool consistency.

FIGS. 5A and 5B show that therapy with rifaximin+NAC decreases systemic inflammation.

FIG. 6 depicts schematic example of a capsule formulation in accordance with various embodiments of the invention described herein. The capsule is not necessarily to scale.

FIG. 7 depicts dissolution of an example of a capsule formulation in accordance with various embodiments of the invention described herein.

FIG. 8 depicts dissolution of NAC minitabs in an example of a capsule formulation in accordance with various embodiments of the invention described herein.

FIG. 9 depicts schematic example of a formulation in accordance with various embodiments of the invention described herein. The formulation is not necessarily to scale.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the invention described herein. Indeed, the invention described herein is in no way limited to the methods and materials described. For purposes of the invention described herein, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, 0.25%, or 0.1% of that referenced numeric indication, if specifically provided for in the claims.

“Substantially” as used herein refers to at least 60%. In various embodiments, where specifically provided for, “substantially” refers to at least 65%, at least 70%, at least 75%, at least 80%, at least 85%, at least 90%, at least 95%, at least 96%, at least 97%, at least 98%, at least 99%, at least 99.5%, at least 99.6%, at least 99.7%, at least 99.8% or at least 99.9%.

“Tablet” as used herein refers to a solid drug form made by compressing one or more active pharmaceutical ingredient, and optionally one or more excipient.

“Mini-tablet” or “minitabs” as used herein refer to a tablet sized to permit a plurality of such tablets to be enclosed within a capsule, such as a size 00 capsule or size 0 capsule. An exemplary mini-tablet can be one having a longest dimension of up to 3 mm.

“Essentially uniform in composition” as used herein refers to uniformity in composition that is at least 95% uniform in composition. In various embodiments, where specifically provided for, “essentially uniform in composition” refers to uniformity in composition that is at least 96%, at least 97%, at least 98%, at least 99%, at least 99.5%, at least 99.6%, at least 99.7%, at least 99.8% or at least 99.9% uniform in composition.

As used herein, a “subject” means a human or animal. Usually, the animal is a vertebrate such as a primate or rodent. Primates include human, chimpanzees, cynomolgus monkeys, spider monkeys, and macaques, e.g., Rhesus. Rodents include mice, rats, woodchucks, ferrets, rabbits and hamsters.

The terms, “patient”, “individual” and “subject” are used interchangeably herein. In an embodiment, the subject is mammal. The mammal may be a human, non-human primate, mouse, rat, dog, cat, horse, or cow, but are not limited to these examples. In some embodiments, the subject is a human.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, whether male or female, are intended to be included within the scope of this term.

A subject may be one who has been previously diagnosed with or identified as suffering from or having a disease, disorder or condition in need of treatment or one or more complications related to the disease, disorder, or condition, and optionally, have already undergone treatment for the disease, disorder, or condition or the one or more complications related to the disease, disorder, or condition. Alternatively, a subject can also be one who has not been previously diagnosed as having a disease, disorder, or condition or one or more complications related to the disease, disorder, or condition. For example, a subject may be one who exhibits one or more risk factors for a disease, disorder, or condition or one or more complications related to the disease, disorder, or condition or a subject who does not exhibit risk factors. A “subject in need” of treatment for a particular disease, disorder, or condition may be a subject suspected of having that disease, disorder, or condition, diagnosed as having that disease, disorder, or condition, already treated or being treated for that disease, disorder, or condition, not treated for that disease, disorder, or condition, or at risk of developing that disease, disorder, or condition.

“Treatment” and “treating,” as used herein refer to both therapeutic treatment and prophylactic or preventative measures, wherein the object is to prevent, slow down and/or lessen the disease even if the treatment is ultimately unsuccessful.

Various embodiments provide for a rifaximin and N-acetylcysteine (NAC) formulation, comprising: a shell comprising a first solid composition comprising an amount of rifaximin and a plurality of a second solid composition comprising an amount of NAC.

Various embodiments provide for a rifaximin and N-acetylcysteine (NAC) formulation, comprising: a shell comprising a first solid composition comprising an amount of rifaximin and a plurality of a coated second solid composition comprising an amount of NAC.

In various embodiments, the amount of rifaximin is about 66 mg. In various embodiments, the amount of rifaximin is about 66.5 mg. In various embodiments, the amount of rifaximin is about 62-70 mg. In various embodiments, the amount of rifaximin is about 59-73 mg.

In various embodiments, the amount of rifaximin is about 132 mg. In various embodiments, the amount of rifaximin is about 133 mg. In various embodiments, the amount of rifaximin is about 125-139 mg. In various embodiments, the amount of rifaximin is about 118-146 mg.

In various embodiments the amount of NAC is about 560 mg. In various embodiments the amount of NAC is about 561.3 mg. In various embodiments, the amount of NAC is about 532-588 mg. In various embodiments, the amount of NAC is about 504-616 mg. In various embodiments, the amount of NAC is about 450-560 mg. In various embodiments, the amount of NAC is about 427-588 mg. In various embodiments, the amount of NAC is about 405-616 mg.

An embodiment provides for a rifaximin and NAC formulation, comprising: a shell comprising a first solid composition comprising about 66 mg of rifaximin and a plurality of a second solid composition comprising about 560 mg of NAC.

An embodiment provides for a rifaximin and NAC formulation, comprising: a shell comprising a first solid composition comprising about 66.5 mg of rifaximin and a plurality of a second solid composition comprising about 561.3 mg of NAC.

An embodiment provides for a rifaximin and NAC formulation, comprising: a shell comprising a first solid composition comprising about 132 mg of rifaximin and a plurality of a second solid composition comprising about 560 mg of NAC.

An embodiment provides for a rifaximin and NAC formulation, comprising: a shell comprising a first solid composition comprising about 133 mg of rifaximin and a plurality of a second solid composition comprising about 561.3 mg of NAC.

An embodiment provides for a rifaximin and NAC formulation, comprising: a shell comprising a first solid composition comprising about 59-73 mg or 62-70 mg of rifaximin and a plurality of a second solid composition comprising about 405-616 mg or 427-588 mg or 450-560 mg or 504-616 mg or 532-588 mg of NAC.

An embodiment provides for a rifaximin and NAC formulation, comprising: a shell comprising a first solid composition comprising about 118-146 mg or 125-139 mg of rifaximin and a plurality of a second solid composition comprising about 405-616 mg or 427-588 mg or 450-560 mg or 504-616 mg or 532-588 mg of NAC.

An embodiment provides for a rifaximin and NAC formulation, comprising: a shell comprising a first solid composition comprising about 66 mg of rifaximin and a plurality of a coated second solid composition comprising about 560 mg of NAC.

An embodiment provides for a rifaximin and NAC formulation, comprising: a shell comprising a first solid composition comprising about 66.5 mg of rifaximin and a plurality of a coated second solid composition comprising about 561.3 mg of NAC.

An embodiment provides for a rifaximin and NAC formulation, comprising: a shell comprising a first solid composition comprising about 132 mg of rifaximin and a plurality of a coated second solid composition comprising about 560 mg of NAC.

An embodiment provides for a rifaximin and NAC formulation, comprising: a shell comprising a first solid composition comprising about 133 mg of rifaximin and a plurality of a coated second solid composition comprising about 561.3 mg of NAC.

An embodiment provides for a rifaximin and NAC formulation, comprising: a shell comprising a first solid composition comprising about 59-73 mg or 62-70 mg of rifaximin and a plurality of a coated second solid composition comprising about 405-616 mg or 427-588 mg or 450-560 mg or 504-616 mg or 532-588 mg of NAC.

An embodiment provides for a rifaximin and NAC formulation, comprising: a shell comprising a first solid composition comprising about 118-146 mg or 125-139 mg of rifaximin and a plurality of a coated second solid composition comprising about 405-616 mg or 427-588 mg or 450-560 mg or 504-616 mg or 532-588 mg of NAC.

In various embodiments, the first solid composition further comprises one or more excipients. In various embodiments, the second solid composition further comprises one or more excipients.

In various embodiments, the first solid composition is in a tablet form.

In various embodiments, herein the second solid composition is in form of a crystal, a pellet, a bead or a mini-tablet. In various embodiments, the crystal, pellet, bead or mini-tablet has a size wherein its longest dimension is 3 mm or less. In various embodiments, the crystal, pellet, bead or mini-tablet has a size wherein its longest dimension is 2 mm or less. In various embodiments, the crystal, pellet, bead or mini-tablet has a size wherein its longest dimension is 1 mm or less.

In various embodiments, the coating of the second solid composition comprises a polymer.

In various embodiments, the coating of the second solid composition comprises at least one polymer that is insoluble at a pH lower than 5. In various embodiments, the coating of the second solid composition comprises at least one polymer that is insoluble at a pH lower than 3.5. In various embodiments, the coating of the second solid composition comprises at least one polymer that is insoluble at a pH between 1.5-3.5.

In various embodiments, the shell does not comprise gelatin. In various embodiments, the shell comprises hypromellose.

In various embodiments, first solid composition comprising the rifaximin is substantially undissolved after 1 hour in a Type 2 dissolution bath containing about 0.1N hydrochloric acid.

In various embodiments, the first solid composition comprising the rifaximin disperses or substantially disburses in fed state simulated intestinal fluid.

Various embodiments provide for a rifaximin and N-acetylcysteine (NAC) formulation, comprising: a shell; a first solid composition comprising an amount of rifaximin; and a plurality of a coated second solid composition comprising an amount of NAC, wherein the first solid composition comprising the rifaximin is a tablet comprising rifaximin and one or more excipients; wherein the plurality of coated second solid composition comprising NAC is a plurality of a mini-tablet comprising NAC and one or more excipients; and wherein the coating comprises at least one polymer that is substantially insoluble at a pH lower than 3.5.

The rifaximin provided in these formulations can be rifaximin base, rifaximin polymorphs (e.g., rifaximin-α), amorphous rifaximin, pharmaceutically acceptable salts thereof, solvates thereof, hydrates thereof, or enantiomers thereof, unless otherwise noted. In particular embodiments, the rifaximin provided in these formulations is rifaximin base. In other particular embodiments, the rifaximin provide in these formulations is polymorph-α. In other particular embodiments, the amorphous form of rifaximin.

As nonlimiting examples, the rifaximin is at least 95%, 96%, 97%, 98%, 99%, 99.5%, 99.6%, 99.7%, 99.8%, or 99.9% in the form that is provided. For example, if rifaximin is provided in the rifaximin-α form, at least 95%, 96%, 97%, 98%, 99%, 99.5%, 99.6%, 99.7%, 99.8%, or 99.9% of the rifaximin will be in the rifaximin-α form. If rifaximin is provided in as rifaximin base, at least 95%, 96%, 97%, 98%, 99%, 99.5%, 99.6%, 99.7%, 99.8%, or 99.9% of the rifaximin will be in the rifaximin base form. In various embodiments, rifaximin base form is not purified for polymorphs.

In various embodiments, the formulation does not comprise 200 mg of rifaximin. In various embodiments, the formulation does not comprise 600 mg of NAC. In various embodiments, the formulation does not comprise 200 mg of rifaximin and 600 mg of NAC.

Various embodiments of the invention provide for a rifaximin and N-acetylcysteine (NAC) formulation, comprising: a solid core composition comprising an amount of rifaximin and an amount of NAC, and a coating around the solid core composition.

In various embodiments, the solid core composition comprises a single layer that is uniform in composition. In various embodiments, the solid core composition comprises a single layer that is essentially uniform in composition.

In various embodiments, the NAC in the solid core composition increases the dissolution of rifaximin relative to a solid core composition without NAC.

In various embodiments, the amount of rifaximin is about 66 mg. In various embodiments, the amount of rifaximin is about 66.5 mg. In various embodiments, the amount of rifaximin is about 62-70 mg. In various embodiments, the amount of rifaximin is about 59-73 mg.

In various embodiments, the amount of rifaximin is about 132 mg. In various embodiments, the amount of rifaximin is about 133 mg. In various embodiments, the amount of rifaximin is about 125-139 mg. In various embodiments, the amount of rifaximin is about 118-146 mg.

In various embodiments the amount of NAC is about 560 mg. In various embodiments the amount of NAC is about 561.3. In various embodiments, the amount of NAC is about 532-588 mg. In various embodiments, the amount of NAC is about 504-616 mg. In various embodiments, the amount of NAC is about 450-560 mg. In various embodiments, the amount of NAC is about 427-588 mg. In various embodiments, the amount of NAC is about 405-616 mg.

An embodiment provides for a rifaximin and NAC formulation, comprising: a solid core composition comprising about 66 mg of rifaximin and about 560 mg of NAC, and a coating around the solid core composition.

An embodiment provides for a rifaximin and NAC formulation, comprising: a solid core composition comprising about 66.5 mg of rifaximin and about 561.3 mg of NAC, and a coating around the solid core composition.

An embodiment provides for a rifaximin and NAC formulation, comprising: a solid core composition comprising about 132 mg of rifaximin and about 560 mg of NAC, and a coating around the solid core composition.

An embodiment provides for a rifaximin and NAC formulation, comprising: a solid core composition comprising about 133 mg of rifaximin and about 561.3 mg of NAC, and a coating around the solid core composition.

An embodiment provides for a rifaximin and NAC formulation, comprising: a solid core composition comprising about 59-73 mg or 62-70 mg of rifaximin and about 405-616 mg or 427-588 mg or 450-560 mg or 504-616 mg or 532-588 mg of NAC, and a coating around the solid core composition.

An embodiment provides for a rifaximin and NAC formulation, comprising: a solid core composition comprising about 118-146 mg or 125-139 mg of rifaximin and about 405-616 mg or 427-588 mg or 450-560 mg or 504-616 mg or 532-588 mg of NAC, and a coating around the solid core composition.

In various embodiments, the solid core composition further comprises one or more excipients.

In various embodiments, the solid core composition is in a tablet form.

In various embodiments, the coating comprises at least one polymer.

In various embodiments, the coating composition comprises at least one polymer that is insoluble at a pH lower than 5. In various embodiments, the coating comprises at least one polymer that is insoluble at a pH lower than 3.5. In various embodiments, the coating comprises at least one polymer that is insoluble at a pH between 1.5-3.5.

In various embodiments, the coating does not comprise gelatin. In various embodiments, the coating comprises hypromellose.

In various embodiments, solid core composition comprising the rifaximin is substantially undissolved after 1 hour in a Type 2 dissolution bath containing about 0.1N hydrochloric acid.

In various embodiments, solid core composition comprising the rifaximin disperses or substantially disperses in fed state simulated intestinal fluid.

In various embodiments, the rifaximin, NAC or both are in granulated forms.

The rifaximin provided in these formulations can be rifaximin base, rifaximin polymorphs (e.g., rifaximin-α), amorphous rifaximin, pharmaceutically acceptable salts thereof, solvates thereof, hydrates thereof, or enantiomers thereof, unless otherwise noted. In particular embodiments, the rifaximin provided in these formulations is rifaximin base. In other particular embodiments, the rifaximin provide in these formulations is rifaximin polymorph-α. In other particular embodiments, rifaximin provide in these formulations is the amorphous form of rifaximin.

As nonlimiting examples, the rifaximin is at least 95%, 96%, 97%, 98%, 99%, 99.5%, 99.6%, 99.7%, 99.8%, or 99.9% in the form that is provided. For example, if rifaximin is provided in the rifaximin-α form, at least 95%, 96%, 97%, 98%, 99%, 99.5%, 99.6%, 99.7%, 99.8%, or 99.9% of the rifaximin will be in the rifaximin-α form. If rifaximin is provided in as rifaximin base, at least 95%, 96%, 97%, 98%, 99%, 99.5%, 99.6%, 99.7%, 99.8%, or 99.9% of the rifaximin will be in the rifaximin base form. In various embodiments, rifaximin base form is not purified for polymorphs.

In various embodiments, the formulation does not comprise 200 mg of rifaximin. In various embodiments, the formulation does not comprise 600 mg of NAC. In various embodiments, the formulation does not comprise 200 mg of rifaximin and 600 mg of NAC.

In various embodiments, the formulations described herein may have sustained-release profiles, i.e., slow release of the active ingredient(s) in the body (e.g., small intestine) over an extended period of time. In various embodiments, the formulations described herein may have a delayed-release profile, i.e. not immediately release the active ingredient(s) upon ingestion; rather, postponement of the release of the active ingredient(s) until the composition is lower in the gastrointestinal tract; for example, for release in the small intestine (e.g., one or more of duodenum, jejunum, ileum) or the large intestine (e.g., one or more of cecum, ascending, transverse, descending or sigmoid portions of the colon, and rectum). For example, a formulation is enteric coated to delay release of the active ingredient(s) until it reaches the small intestine or large intestine.

In various embodiments, the formulation releases NAC and rifaximin in the small intestine. In various embodiments, the formulation of the invention described herein releases at least 5%, 10%, 15%, 20%, 25%, 30%, 35%, 40%, 45%, 50%, 55%, or 60% of the NAC and rifaximin in the small intestine. For example, the formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the NAC and rifaximin in the small intestine.

In one embodiment, the formulation releases the NAC and rifaximin in the duodenum. In various embodiments, the formulation of the invention described herein releases at least 5%, 10%, 15%, 20%, 25%, 30%, 35%, 40%, 45%, 50%, 55%, or 60% of the NAC and rifaximin in the duodenum. For example, the formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the NAC and rifaximin in the duodenum.

In another embodiment, the formulation releases the NAC and rifaximin in the jejunum. In various embodiments, the formulation of the invention described herein releases at least 5%, 10%, 15%, 20%, 25%, 30%, 35%, 40%, 45%, 50%, 55%, or 60% of the NAC and rifaximin in the jejunum. For example, the formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the NAC and rifaximin in the jejunum.

In a further embodiment, the formulation releases the NAC and rifaximin in the ileum and/or the ileocecal junction. In various embodiments, the formulation of the invention described herein releases at least 5%, 10%, 15%, 20%, 25%, 30%, 35%, 40%, 45%, 50%, 55%, or 60% of the NAC and rifaximin in the ileum and/or the ileocecal junction. For example, the formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the NAC and rifaximin in the ileum and/or the ileocecal junction.

In certain embodiments, the formulation does not substantially release the NAC and rifaximin in the stomach.

In certain embodiments, the formulation releases the NAC and rifaximin at a specific pH. For example, in some embodiments, the formulation is substantially non-dispersible in an acidic environment and substantially dispersible (e.g., dissolves rapidly) in a near neutral to alkaline environment. In some embodiments, stability is indicative of not substantially releasing while instability is indicative of substantially releasing. For example, in some embodiments, the formulation is substantially non-dispersible at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In some embodiments, the formulations are non-dispersible in lower pH areas and therefore do not substantially release in, for example, the stomach. In some embodiments, formulation is substantially non-dispersible at a pH of about 1 to about 4 or lower and substantially dispersible at pH values that are greater. In these embodiments, the formulation is not substantially released in the stomach. In these embodiments, the formulation is substantially released in the small intestine (e.g., one or more of the duodenum, jejunum, and ileum). In various embodiments, the pH values recited herein may be adjusted as known in the art to account for the state of the subject, e.g., whether in a fasting or postprandial state.

In some embodiments, the formulation is substantially non-dispersible in gastric fluid and substantially dispersible in intestinal fluid and, accordingly, is substantially released in the small intestine (e.g., one or more of the duodenum, jejunum, and ileum).

In some embodiments, the formulation is non-dispersible in gastric fluid or non-dispersible in acidic environments. These formulations release about 30% or less by weight of the NAC and rifaximin in the formulation in gastric fluid with a pH of about 4 to about 5 or less than 4 or less than 5, or simulated gastric fluid with a pH of about 4 to about 5 or less than 4 or less than 5, in about 15, or about 30, or about 45, or about 60, or about 90 minutes. Formulations of the invention may release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the NAC and rifaximin in the formulation in gastric fluid with a pH of 4-5, or less than 4 or less than 5, or simulated gastric fluid with a pH of 4-5 or less than 4, or less than 5, in about 15, or about 30, or about 45, or about 60, or about 90 minutes. Formulations of the invention may release about 1%, about 2%, about 3%, about 4%, about 5%, about 6%, about 7%, about 8%, about 9%, or about 10% by weight of the total NAC and rifaximin in the formulation in gastric fluid with a pH of 5 or less, or simulated gastric fluid with a pH of 5 or less, in about 15, or about 30, or about 45, or about 60, or about 90 minutes.

In some embodiments, the formulation is dispersible in intestinal fluid. These formulations release about 70% or more by weight of the NAC and rifaximin in the formulation in intestinal fluid or simulated intestinal fluid in about 15, or about 30, or about 45, or about 60, or about 90 minutes. In some embodiments, the formulation is dispersible in near neutral to alkaline environments. These formulations release about 70% or more by weight of NAC and rifaximin in the formulation in intestinal fluid with a pH of about 4-5 or greater than 4, or greater than 5, or simulated intestinal fluid with a pH of about 4-5 or greater than 4, or greater than 5, in about 15, or about 30, or about 45, or about 60, or about 90 minutes. A formulation that is dispersible in near neutral or alkaline environments may release 70% or more by weight of NAC and rifaximin in the formulation in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

In one embodiment, the formulation may remain essentially intact, or may be essentially insoluble, in gastric fluid. The stability of the delayed-release coating can be pH dependent. Delayed-release coatings that are pH dependent will be substantially non-dispersible in acidic environments (pH of about 5 or less), and substantially dispersible in near neutral to alkaline environments (pH greater than about 5). For example, the delayed-release coating may essentially disintegrate or dissolve in near neutral to alkaline environments such as are found in the small intestine (e.g., one or more of the duodenum, jejunum, and ileum).

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

Alternatively, the stability of the formulation can be enzyme-dependent. Delayed-release coatings that are enzyme dependent will be substantially non-dispersible in fluid that does not contain a particular enzyme and substantially dispersible in fluid containing the enzyme. The delayed-release coating will essentially disintegrate or dissolve in fluid containing the appropriate enzyme. Enzyme-dependent control can be brought about, for example, by using materials which release the active ingredient only on exposure to enzymes in the intestine, such as galactomannans. Also, the stability of the formulation can be dependent on enzyme stability in the presence of a microbial enzyme present in the gut flora.

### Carriers, Excipients, Coatings and Salts

The compositions and formulations of the invention described herein may further comprise a pharmaceutically acceptable carrier or excipient. As one skilled in the art will recognize, the formulations can be in any suitable form appropriate for the desired use and route of administration. Examples of suitable dosage forms include, for example, oral and parenteral dosage forms.

Suitable dosage forms for oral use include, for example, solid dosage forms. In another embodiment, the formulation is in the form of a capsule. In yet another embodiment, the formulation is in the form of a soft-gel capsule. In some embodiments, the shell does not comprise gelatin. In a further embodiment, the formulation is in the form of a hydroxypropyl methylcellulose (HPMC) (also known as hypromellose) capsule.

In another embodiment, the formulation is in the form of a coated tablet. In yet another embodiment, the formulation is in the form of a soft-gel tablet. In some embodiments, the coating does not comprise gelatin. In a further embodiment, the formulation is coated with hydroxypropyl methylcellulose (HPMC) (also known as hypromellose).

In these various dosage forms, the active compound can be mixed with at least one inert, pharmaceutically acceptable excipient or carrier such as sodium citrate, dicalcium phosphate, etc., and/or a) fillers, diluents, or extenders such as starches, lactose, sucrose, glucose, mannitol, silicic acid, microcrystalline cellulose (e.g., Avicel PH102), and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose, hydroxymethyl cellulose, and copovidones such as Kollidon® VA64, and Kollidon® VA64 Fine, etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., i) lubricants such as talc, calcium stearate, magnesium stearate, solid polyethylene glycols, sodium lauryl sulfate, glyceryl behenate, etc., j) antioxidants such as propyl gallate, butylated hydroxyanisole (BHA), butylated hydroxytoluene (BHT), ethylenediaminetetraacetic acid (also known as Edetic Acid or EDTA) etc., k) viscosity and dispersion agents such as silicon dioxide or silica, and mixtures of such excipients. One of skill in the art will recognize that particular excipients may have two or more functions in the oral dosage form. In cases of an oral dosage forms, for example, a capsule or a tablet, the dosage form may also comprise buffering agents.

The compositions and modified release formulations can additionally include a surface active agent. Surface active agents suitable for use in the invention described herein include, but are not limited to, any pharmaceutically acceptable, non-toxic surfactant. Classes of surfactants suitable for use in the compositions of the invention include, but are not limited to polyethoxylated fatty acids, PEG-fatty acid diesters, PEG-fatty acid mono-and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono-and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In some embodiments, compositions of the invention may comprise one or more surfactants including, but not limited to, sodium lauryl sulfate, polysorbate 20, polysorbate 40, polysorbate 60, polysorbate 80, and triethyl citrate.

The compositions and formulations can also contain pharmaceutically acceptable plasticizers to obtain the desired mechanical properties such as flexibility and hardness. Such plasticizers include, but are not limited to, triacetin, citric acid esters, phthalic acid esters, dibutyl sebacate, cetyl alcohol, polyethylene glycols, polysorbates or other plasticizers.

The compositions and formulations can also include one or more application solvents. Some of the more common solvents that can be used to apply, for example, a delayed-release coating composition include isopropyl alcohol, acetone, methylene chloride and the like.

The compositions and formulations can also include one or more disintegrants. Illustrative disintegrants that may be utilized include, but are not limited to crospovidones such as Kollidon® CL, Kollidon® CL-F, Kollidon® CL-SF, or Kollidon® CL-M,

The compositions and formulations can also include one or more alkaline materials. Alkaline material suitable for use in compositions of the invention include, but are not limited to, sodium, potassium, calcium, magnesium and aluminum salts of acids such as phosphoric acid, carbonic acid, citric acid and other aluminum/magnesium compounds. In addition, the alkaline material may be selected from antacid materials such as aluminum hydroxides, calcium hydroxides, magnesium hydroxides and magnesium oxide.

The solid oral dosage forms can be prepared by any conventional method known in the art, for example granulation (e.g., wet or dry granulation) of the active compound (e.g., NAC and rifaximin) with one or more suitable excipients. Alternatively, the active compound can be layered onto an inert core (e.g., a nonpareil/sugar sphere or silica sphere) using conventional methods such as fluidized bed or pan coating, or extruded and spheronized using methods known in the art, into active compound-containing beads. Such beads can then be incorporated into tablets or capsules using conventional methods.

Besides inert diluents, the oral compositions can also include adjuvants such as sweetening, flavoring, and perfuming agents.

Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

The compositions and formulations comprising the NAC and rifaximin of the invention described herein may be presented in unit dosage forms and may be prepared by any of the methods known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, the compositions and formulations of the invention described herein may utilize one or more coatings such as delayed-release coatings to provide for effective, delayed yet substantial delivery of the NAC and rifaximin to the specific areas of GI tract.

In one embodiment, the delayed-release coating includes an enteric agent that is substantially non-dispersible in acidic environments and substantially dispersible in near neutral to alkaline environments. In an embodiment, the delayed-release coating contains an enteric agent that is substantially non-dispersible in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The EUDRAGIT®-type polymers include, for example, EUDRAGIT® FS 30D, L 30 D-55, L 100-55, L 100, L 12,5, L 12,5 P, RL 30 D, RL PO, RL 100, RL 12,5, RS 30 D, RS PO, RS 100, RS 12,5, NE 30 D, NE 40 D, NM 30 D, S 100, S 12,5, and S 12,5 P. Similar polymers include Kollicoat® MAE 30 DP and Kollicoat® MAE 100 P. In some embodiments, one or more of EUDRAGIT® FS 30D, L 30 D-55, L 100-55, L 100, L 12,5, L 12,5 P RL 30 D, RL PO, RL 100, RL 12,5, RS 30 D, RS PO, RS 100, RS 12,5, NE 30 D, NE 40 D, NM 30 D, S 100, S 12,5 S 12,5 P, Kollicoat® MAE 30 DP and Kollicoat® MAE 100 P is used. In various embodiments, the enteric agent may be a combination of the foregoing solutions or dispersions. In certain embodiments, one or more coating system additives are used with the enteric agent. For example, one or more PlasACRYL™ additives may be used as an anti-tacking agent coating additive. Illustrative PlasACRYL™ additives include, but are not limited to PlasACRYL™ HTP20 and PlasACRYL™ T20. In an embodiment, PlasACRYL™ HTP20 is formulated with EUDRAGIT® L 30 D-55 coatings. In another embodiment, PlasACRYL™ T20 is formulated with EUDRAGIT® FS 30 D coatings.

In another embodiment, the delayed-release coating may degrade as a function of time when in aqueous solution without regard to the pH and/or presence of enzymes in the solution. Such a coating may comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings may be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers useful in the invention described herein include polyvinyl esters, polyvinyl acetals, polyacrylic acid esters, butadiene styrene copolymers, and the like. In one embodiment, colonic delivery is achieved by use of a slowly-eroding wax plug (e.g., various PEGS, including for example, PEG6000).

In a further embodiment, the delayed-release coating may be degraded by a microbial enzyme present in the gut flora. In one embodiment, the delayed-release coating may be degraded by a bacteria present in the small intestine.

Furthermore, in various embodiments, the agents described herein may be in the form of a pharmaceutically acceptable salt, namely those salts which are suitable for use in contact with the tissues of humans and other animals without undue toxicity, irritation, allergic response and the like, and are commensurate with a reasonable benefit/risk ratio. Pharmaceutically acceptable salts are well known in the art. The salts can be prepared in situ during the final isolation and purification of the therapeutic agents, or separately by reacting the free base function with a suitable acid or a free acid functionality with an appropriate alkaline moiety. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

### Kits

Various embodiments of the invention described herein provide for a kit comprising a formulation of the invention described herein. In various embodiments, the kit further comprises instructions to for using the formulation comprising NAC and rifaximin.

The kit is an assemblage of materials or components, including at least one of the inventive compositions. Thus, in some embodiments the kit contains a composition including NAC and rifaximin as described herein.

Various embodiments provide for a kit comprises a formulation comprising: rifaximin and N-acetylcysteine (NAC) wherein the formulation is provided in one or more doses for a total of about 198 mg of rifaximin per day and about 1680 mg of N-acetylcysteine (NAC) per day. In various embodiments the kit comprises dosages for 5-10 days. In various embodiments the kit comprises dosages for 7-14 days. In various embodiments the kit comprises dosages for 7-10 days. In various embodiments, the kit can comprise dosages for chronic treatment; for example, doses for 30 days, 60 days, or 90 days at a time.

Various embodiments provide for a kit comprises a formulation comprising: rifaximin and N-acetylcysteine (NAC) wherein the formulation is provided in one or more doses for a total of about 198 mg of rifaximin per day and about 1350 mg to about 1680 mg of N-acetylcysteine (NAC) per day. In various embodiments the kit comprises dosages for 5-10 days. In various embodiments the kit comprises dosages for 7-14 days. In various embodiments the kit comprises dosages for 7-10 days. In various embodiments, the kit can comprise dosages for chronic treatment; for example, doses for 30 days, 60 days, or 90 days at a time.

Various embodiments provide for a kit comprises a formulation comprising: rifaximin and N-acetylcysteine (NAC) wherein the formulation is provided in one or more doses for a total of about 186-210 mg of rifaximin per day and about 1350 mg to about 1680 mg of N-acetylcysteine (NAC) per day. In various embodiments the kit comprises dosages for 7-14 days. In various embodiments the kit comprises dosages for 5-10 days. In various embodiments the kit comprises dosages for 7-10 days. In various embodiments, the kit can comprise dosages for chronic treatment; for example, doses for 30 days, 60 days, or 90 days at a time.

Various embodiments provide for a kit comprises a formulation comprising: rifaximin and N-acetylcysteine (NAC) wherein the formulation is provided in one or more doses for a total of about 177-219 mg of rifaximin per day and 1350 mg to about 1680 mg of N-acetylcysteine (NAC) per day. In various embodiments the kit comprises dosages for 5-10 days. In various embodiments the kit comprises dosages for 7-14 days. In various embodiments the kit comprises dosages for 7-10 days. In various embodiments, the kit can comprise dosages for chronic treatment; for example, doses for 30 days, 60 days, or 90 days at a time.

Various embodiments provide for a kit comprises a formulation comprising: rifaximin and N-acetylcysteine (NAC) wherein the formulation is provided in one or more doses for a total of about 396 mg of rifaximin per day and about 1680 mg of N-acetylcysteine (NAC) per day. In various embodiments the kit comprises dosages for 5-10 days. In various embodiments the kit comprises dosages for 7-14 days. In various embodiments the kit comprises dosages for 7-10 days. In various embodiments, the kit can comprise dosages for chronic treatment; for example, doses for 30 days, 60 days, or 90 days at a time.

Various embodiments provide for a kit comprises a formulation comprising: rifaximin and N-acetylcysteine (NAC) wherein the formulation is provided in one or more doses for a total of about 396 mg of rifaximin per day and 1350 mg to about 1680 mg of N-acetylcysteine (NAC) per day. In various embodiments the kit comprises dosages for 5-10 days. In various embodiments the kit comprises dosages for 7-14 days. In various embodiments the kit comprises dosages for 7-10 days. In various embodiments, the kit can comprise dosages for chronic treatment; for example, doses for 30 days, 60 days, or 90 days at a time.

Various embodiments provide for a kit comprises a formulation comprising: rifaximin and N-acetylcysteine (NAC) wherein the formulation is provided in one or more doses for a total of about 375-417 mg of rifaximin per day and 1350 mg to about 1680 mg of N-acetylcysteine (NAC) per day. In various embodiments the kit comprises dosages for 5-10 days. In various embodiments the kit comprises dosages for 7-14 days. In various embodiments the kit comprises dosages for 7-10 days. In various embodiments, the kit can comprise dosages for chronic treatment; for example, doses for 30 days, 60 days, or 90 days at a time.

Various embodiments provide for a kit comprises a formulation comprising: rifaximin and N-acetylcysteine (NAC) wherein the formulation is provided in one or more doses for a total of about 354-438 mg of rifaximin per day and 1350 mg to about 1680 mg of N-acetylcysteine (NAC) per day. In various embodiments the kit comprises dosages for 5-10 days. In various embodiments the kit comprises dosages for 7-14 days. In various embodiments the kit comprises dosages for 7-10 days. In various embodiments, the kit can comprise dosages for chronic treatment; for example, doses for 30 days, 60 days, or 90 days at a time.

The rifaximin provided in these formulations can be rifaximin base, rifaximin polymorphs (e.g., rifaximin-α), amorphous rifaximin, pharmaceutically acceptable salts thereof, solvates thereof, hydrates thereof, or enantiomers thereof, unless otherwise noted. In particular embodiments, the rifaximin provided in these formulations is rifaximin base. In other particular embodiments, the rifaximin provide in these formulations is polymorph-α. In other particular embodiments, the rifaximin provide in these formulations is the amorphous form of rifaximin.

As nonlimiting examples, the rifaximin is at least 95%, 96%, 97%, 98%, 99%, 99.5%, 99.6%, 99.7%, 99.8%, or 99.9% in the form that is provided. For example, if rifaximin is provided in the rifaximin-α form, at least 95%, 96%, 97%, 98%, 99%, 99.5%, 99.6%, 99.7%, 99.8%, or 99.9% of the rifaximin will be in the rifaximin-α form. If rifaximin is provided in as rifaximin base, at least 95%, 96%, 97%, 98%, 99%, 99.5%, 99.6%, 99.7%, 99.8%, or 99.9% of the rifaximin will be in the rifaximin base form. In various embodiments, rifaximin base form is not purified for polymorphs.

The exact nature of the components configured in the inventive kit depends on its intended purpose. For example, some embodiments are configured for the purpose of treating irritable bowel syndrome (IBS), IBS-D, or H₂S positive IBS; other embodiments are configured for treating SIBO; still other embodiments are configured for decreasing Escherichia coli (E. Coli) load, Klebsiella load, and total bacterial load in the small bowel, or configured for improving stool consistency, reducing systemic inflammation, or both.

In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat IBS, treat H₂S positive IBS, treat D-IBS, treat SIBO, decrease E. Coli load, Klebsiella load, and total bacterial load in the small bowel, or to improve stool consistency, reducing systemic inflammation, or both. Optionally, the kit also contains other useful components, such as, diluents, pharmaceutically acceptable carriers, syringes, pipetting or measuring tools, or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in treating gastrointestinal diseases. As used herein, the term “package” refers to a suitable solid matrix or material such as glass, plastic, paper, foil, and the like, capable of holding the individual kit components. Thus, for example, a package can be a bottle used to contain suitable quantities of the formulation containing NAC and rifaximin described herein. The packaging material generally has an external label which indicates the contents and/or purpose of the kit and/or its components.

### Therapeutic Methods

Various embodiments of the invention provide for treating irritable bowel syndrome (IBS), comprising: administering one or more doses of a rifaximin and N-acetylcysteine (NAC) formulation as described herein to a subject in need thereof. In various embodiments, the IBS is diarrhea predominant IBS (IBS-D). In various embodiments, the IBS is H₂S positive IBS. That is, the subject tested positive for hydrogen sulfide.

Various embodiments of the invention provide for treating small intestinal bacterial overgrowth (SIBO), comprising: administering one or more doses of a rifaximin and N-acetylcysteine (NAC) formulation described herein to a subject in need thereof.

Various embodiments of the invention provide for decreasing Escherichia coli (E. Coli) load, Klebsiella load, and total bacterial load in the small bowel, comprising: administering one or more doses of a rifaximin and N-acetylcysteine (NAC) formulation described herein to a subject in need thereof. In various embodiments, the decrease in the E. Coli load, Klebsiella load, and the total bacterial load occurs in the ileum. In various embodiments, the decrease in the E. Coli load, Klebsiella load, and the total bacterial load occurs in the duodenum.

Various embodiments of the invention provide for method of improving stool consistency, comprising: administering one or more doses of a rifaximin and N-acetylcysteine (NAC) formulation described herein to a subject in need thereof.

Various embodiments of the invention provide for method of reducing systemic inflammation, comprising: administering one or more doses of a rifaximin and N-acetylcysteine (NAC) formulation described herein to a subject in need thereof.

Various embodiments of the invention provide for method of improving stool consistency and reducing systemic inflammation, comprising: administering one or more doses of a rifaximin and N-acetylcysteine (NAC) formulation described herein to a subject in need thereof.

In various embodiments, the subject tested positive for H₂S, for example, based on a breath test. Thus, the subject may have an H₂S positive condition. Methods of testing for H₂S are known in the art. Methods of testing for H₂S are also described in International Application No. PCT/US2018/019490 filed Feb. 23, 2018, the contents of which is herein incorporated by reference in their entirety as though fully set forth.

The subject can be a mammalian subject, and preferably a human subject.

In various embodiments, administering the one or more doses of the rifaximin and N-acetylcysteine (NAC) formulation is via oral administration.

In various embodiments, administering one or more doses of the rifaximin and N-acetylcysteine (NAC) formulation comprises administering two or more doses of the formulation per day to the subject in need thereof. In various embodiments, administering one or more doses of the rifaximin and N-acetylcysteine (NAC) formulation comprises administering three dosages per day to the subject in need thereof.

In various embodiments, administering one or more doses of the rifaximin and N-acetylcysteine (NAC) formulation comprises administering the one or more doses for about 7 to 14 days. administering one or more doses of the rifaximin and N-acetylcysteine (NAC) formulation comprises administering the one or more doses for about 7 to 10 days. In various embodiments, administering one or more doses of the rifaximin and N-acetylcysteine (NAC) formulation comprises administering the one or more doses for about 10 to 14 days. administering one or more doses of the rifaximin and N-acetylcysteine (NAC) formulation comprises administering the one or more doses for about 5 to 10 days.

In various embodiments, administering one or more doses of the rifaximin and N-acetylcysteine (NAC) formulation comprises administering one dose per day for about 7 to 14 days to the subject in need thereof. In various embodiments, administering one or more doses of the rifaximin and N-acetylcysteine (NAC) formulation comprises administering one dose per day for about 7 to 10 days to the subject in need thereof. In various embodiments, administering one or more doses of the rifaximin and N-acetylcysteine (NAC) formulation comprises administering one dose per day for about 10 to 14 days to the subject in need thereof. In various embodiments, administering one or more doses of the rifaximin and N-acetylcysteine (NAC) formulation comprises administering one dose per day for about 5 to 10 days to the subject in need thereof.

In various embodiments, administering one or more doses of the rifaximin and N-acetylcysteine (NAC) formulation comprises administering two dosages per day for about 7 to 14 days to the subject in need thereof. In various embodiments, administering one or more doses of the rifaximin and N-acetylcysteine (NAC) formulation comprises administering two dosages per day for about 7 to 10 days to the subject in need thereof. In various embodiments, administering one or more doses of the rifaximin and N-acetylcysteine (NAC) formulation comprises administering two dosages per day for about 10 to 14 days to the subject in need thereof. In various embodiments, administering one or more doses of the rifaximin and N-acetylcysteine (NAC) formulation comprises administering two dosages per day for about 5 to 10 days to the subject in need thereof.

In various embodiments, administering one or more doses of the rifaximin and N-acetylcysteine (NAC) formulation comprises administering three dosages per day for about 7 to 14 days to the subject in need thereof. In various embodiments, administering one or more doses of the rifaximin and N-acetylcysteine (NAC) formulation comprises administering three dosages per day for about 7 to 10 days to the subject in need thereof. In various embodiments, administering one or more doses of the rifaximin and N-acetylcysteine (NAC) formulation comprises administering three dosages per day for about 10 to 14 days to the subject in need thereof. In various embodiments, administering one or more doses of the rifaximin and N-acetylcysteine (NAC) formulation comprises administering three dosages per day for about 5 to 10 days to the subject in need thereof.

In various embodiments, administering one or more doses of the rifaximin and N-acetylcysteine (NAC) formulation comprises administering four dosages per day for about 7 to 14 days to the subject in need thereof. In various embodiments, administering one or more doses of the rifaximin and N-acetylcysteine (NAC) formulation comprises administering four dosages per day for about 7 to 10 days to the subject in need thereof. In various embodiments, administering one or more doses of the rifaximin and N-acetylcysteine (NAC) formulation comprises administering four dosages per day for about 10 to 14 days to the subject in need thereof. In various embodiments, administering one or more doses of the rifaximin and N-acetylcysteine (NAC) formulation comprises administering four dosages per day for about 5 to 10 days to the subject in need thereof.

In various embodiments, administering one or more doses of the rifaximin and N-acetylcysteine (NAC) formulation comprises administering 1, 2, 3 or 4 doses per day for chronic treatment. Chronic treatment can be treatment that lasts for about 30 days, about 60 days, about 90 days, about 120 days, about 150 days, about 180 days, or more than 180 days. In various embodiments, chronic treatment can be treatment that lasts for about 15-30 days, 30-60 days, about 60-90 days, about 90-120 days, or about 120-180 days. Chronic treatment can also be cycles of treatment; for example, administering treatment for a certain number of days and then a period time of no treatment with rifaximin. An example of a cycle includes but is not limited to administering treatment for about 10 days and no treatment for about 1-3 months afterwards. Thereafter, a second cycle can be administering treatment for another 10 days and no treatment for another 1-3 months. Another example of a cycle can be administering treatment for about 14 days and then no treatment for about 1-3 months.

Administering the formulations in accordance with embodiments of the invention can be achieved by oral administration.

In various embodiments, the rifaximin and N-acetylcysteine (NAC) formulation administered in accordance with these methods comprises: a shell comprising a first solid composition comprising an amount of rifaximin and a plurality of a second solid composition comprising an amount of NAC.

In various embodiments, the rifaximin and N-acetylcysteine (NAC) formulation administered in accordance with these methods comprises: a shell comprising a first solid composition comprising an amount of rifaximin and a plurality of a coated second solid composition comprising an amount of NAC.

In various embodiments, the amount of rifaximin is about 66 mg. In various embodiments, the amount of rifaximin is about 66.5 mg. In various embodiments, the amount of rifaximin is about 62-70 mg. In various embodiments, the amount of rifaximin is about 59-73 mg.

In various embodiments, the amount of rifaximin is about 132 mg. In various embodiments, the amount of rifaximin is about 133 mg. In various embodiments, the amount of rifaximin is about 125-139 mg. In various embodiments, the amount of rifaximin is about 118-146 mg.

In various embodiments the amount of NAC is about 560 mg. In various embodiments the amount of NAC is about 561.3 In various embodiments, the amount of NAC is about 532-588 mg. In various embodiments, the amount of NAC is about 504-616 mg. In various embodiments, the amount of NAC is about 450-560 mg. In various embodiments, the amount of NAC is about 427-588 mg. In various embodiments, the amount of NAC is about 405-616 mg.

In various embodiments, the rifaximin and N-acetylcysteine (NAC) formulation administered in accordance with these methods comprises a shell comprising a first solid composition comprising about 66 mg of rifaximin and a plurality of a second solid composition comprising about 560 mg of NAC.

In various embodiments, the rifaximin and N-acetylcysteine (NAC) formulation administered in accordance with these methods comprises a shell comprising a first solid composition comprising about 66.5 mg of rifaximin and a plurality of a second solid composition comprising about 561.3 mg of NAC.

In various embodiments, the rifaximin and N-acetylcysteine (NAC) formulation administered in accordance with these methods comprises a shell comprising a first solid composition comprising about 132 mg of rifaximin and a plurality of a second solid composition comprising about 560 mg of NAC.

In various embodiments, the rifaximin and N-acetylcysteine (NAC) formulation administered in accordance with these methods comprises a shell comprising a first solid composition comprising about 133 mg of rifaximin and a plurality of a second solid composition comprising about 561.3 mg of NAC.

In various embodiments, the rifaximin and N-acetylcysteine (NAC) formulation administered in accordance with these methods comprises a shell comprising a first solid composition comprising about 59-73 mg or 62-70 mg of rifaximin and a plurality of a second solid composition comprising about 405-616 mg or 427-588 mg or 450-560 mg or 504-616 mg or 532-588 mg of NAC.

In various embodiments, the rifaximin and N-acetylcysteine (NAC) formulation administered in accordance with these methods comprises a shell comprising a first solid composition comprising about 118-146 mg or 125-139 mg of rifaximin and a plurality of a second solid composition comprising about 405-616 mg or 427-588 mg or 450-560 mg or 504-616 mg or 532-588 mg of NAC.

In various embodiments, the rifaximin and N-acetylcysteine (NAC) formulation administered in accordance with these methods comprises a shell comprising a first solid composition comprising about 66 mg of rifaximin and a plurality of a coated second solid composition comprising about 560 mg of NAC.

In various embodiments, the rifaximin and N-acetylcysteine (NAC) formulation administered in accordance with these methods comprises a shell comprising a first solid composition comprising about 66.5 mg of rifaximin and a plurality of a coated second solid composition comprising about 561.3 mg of NAC.

In various embodiments, the rifaximin and N-acetylcysteine (NAC) formulation administered in accordance with these methods comprises a shell comprising a first solid composition comprising about 132 mg of rifaximin and a plurality of a coated second solid composition comprising about 560 mg of NAC.

In various embodiments, the rifaximin and N-acetylcysteine (NAC) formulation administered in accordance with these methods comprises a shell comprising a first solid composition comprising about 133 mg of rifaximin and a plurality of a coated second solid composition comprising about 561.3 mg of NAC.

In various embodiments, the rifaximin and N-acetylcysteine (NAC) formulation administered in accordance with these methods comprises a shell comprising a first solid composition comprising about 59-73 mg or 62-70 mg of rifaximin and a plurality of a coated second solid composition comprising about 405-616 mg or 427-588 mg or 450-560 mg or 504-616 mg or 532-588 mg of NAC.

In various embodiments, the rifaximin and N-acetylcysteine (NAC) formulation administered in accordance with these methods comprises a shell comprising a first solid composition comprising about 118-146 mg or 125-139 mg of rifaximin and a plurality of a coated second solid composition comprising about 405-616 mg or 427-588 mg or 450-560 mg or 504-616 mg or 532-588 mg of NAC.

In various embodiments, the first solid composition in the rifaximin and N-acetylcysteine (NAC) formulation further comprises one or more excipients. In various embodiments, the second solid composition in the rifaximin and N-acetylcysteine (NAC) formulation further comprises one or more excipients.

In various embodiments, the first solid composition in the rifaximin and N-acetylcysteine (NAC) formulation is in a tablet form.

In various embodiments, the second solid composition in the rifaximin and N-acetylcysteine (NAC) formulation is in form of a crystal, a pellet, a bead or a mini-tablet. In various embodiments, the crystal, pellet, bead or mini-tablet has a size wherein its longest dimension is 3 mm or less. In various embodiments, the crystal, pellet, bead or mini-tablet has a size wherein its longest dimension is 2 mm or less. In various embodiments, the crystal, pellet, bead or mini-tablet has a size wherein its longest dimension is up to 1 mm.

In various embodiments, the coating of the second solid composition comprises a polymer.

In various embodiments, the coating of the second solid composition comprises at least one polymer that is insoluble at a pH lower than 5. In various embodiments, the coating of the second solid composition comprises at least one polymer that is insoluble at a pH lower than 3.5. In various embodiments, the coating of the second solid composition comprises at least one polymer that is insoluble at a pH between 1.5-3.5.

In various embodiments, the shell does not comprise gelatin. In various embodiments, the shell comprises hypromellose.

In various embodiments, first solid composition comprising the rifaximin is substantially undissolved after 1 hour in a Type 2 dissolution bath containing about 0.1N hydrochloric acid.

In various embodiments, the first solid composition comprising the rifaximin entirely disperses or substantially disburses in fed state simulated intestinal fluid.

In various embodiments of these methods a rifaximin and N-acetylcysteine (NAC) formulation is orally administered to the subject in need thereof, the formulation comprises: a shell comprising a first solid composition comprising an amount of rifaximin and a plurality of a coated second solid composition comprising an amount of NAC, wherein the first solid composition comprising the rifaximin is a tablet comprising rifaximin and one or more excipients, and wherein the plurality of coated second solid composition comprising NAC is a plurality of a mini-tablet comprising NAC and one or more excipients; and wherein the coating comprises at least one polymer that is substantially insoluble at a pH lower than 3.5.

The rifaximin provided in these formulations can be rifaximin base, rifaximin polymorphs (e.g., rifaximin-α), amorphous rifaximin, pharmaceutically acceptable salts thereof, solvates thereof, hydrates thereof, or enantiomers thereof, unless otherwise noted. In particular embodiments, the rifaximin provided in these formulations is rifaximin base. In other particular embodiments, the rifaximin provide in these formulations is polymorph-α. In other particular embodiments, the rifaximin provide in these formulations is the amorphous form of rifaximin.

As nonlimiting examples, the rifaximin is at least 95%, 96%, 97%, 98%, 99%, 99.5%, 99.6%, 99.7%, 99.8%, or 99.9% in the form that is provided. For example, if rifaximin is provided in the rifaximin-α form, at least 95%, 96%, 97%, 98%, 99%, 99.5%, 99.6%, 99.7%, 99.8%, or 99.9% of the rifaximin will be in the rifaximin-α form. If rifaximin is provided in as rifaximin base, at least 95%, 96%, 97%, 98%, 99%, 99.5%, 99.6%, 99.7%, 99.8%, or 99.9% of the rifaximin will be in the rifaximin base form. In various embodiments, rifaximin base form is not purified for polymorphs.

In various embodiments, the amount of rifaximin in the rifaximin and NAC formulation administered is not 200 mg. In various embodiments, the amount of NAC in the rifaximin and NAC formulation is not 600 mg of NAC. In various embodiments, the amount of rifaximin and the amount of NAC in the rifaximin and NAC formulation administered is not 200 mg of rifaximin and 600 mg of NAC.

In various embodiments, the rifaximin and N-acetylcysteine (NAC) formulation administered in accordance with these methods comprises: a solid core composition comprising an amount of rifaximin and an amount of NAC, and a coating around the solid core composition.

In various embodiments, the amount of rifaximin is about 66 mg. In various embodiments, the amount of rifaximin is about 66.5 mg. In various embodiments, the amount of rifaximin is about 62-70 mg. In various embodiments, the amount of rifaximin is about 59-73 mg.

In various embodiments, the amount of rifaximin is about 132 mg. In various embodiments, the amount of rifaximin is about 133 mg. In various embodiments, the amount of rifaximin is about 125-139 mg. In various embodiments, the amount of rifaximin is about 118-146 mg.

In various embodiments the amount of NAC is about 560 mg. In various embodiments the amount of NAC is about 561.3. In various embodiments, the amount of NAC is about 532-588 mg. In various embodiments, the amount of NAC is about 504-616 mg. In various embodiments, the amount of NAC is about 450-560 mg. In various embodiments, the amount of NAC is about 427-588 mg. In various embodiments, the amount of NAC is about 405-616 mg.

In various embodiments, the rifaximin and N-acetylcysteine (NAC) formulation administered in accordance with these methods comprises a solid core composition comprising about 66 mg of rifaximin and about 560 mg of NAC, and a coating around the solid core composition.

In various embodiments, the rifaximin and N-acetylcysteine (NAC) formulation administered in accordance with these methods comprises a solid core composition comprising about 66.5 mg of rifaximin and about 561.3 mg of NAC, and a coating around the solid core composition.

In various embodiments, the rifaximin and N-acetylcysteine (NAC) formulation administered in accordance with these methods comprises a solid core composition comprising about 132 mg of rifaximin and about 560 mg of NAC.

In various embodiments, the rifaximin and N-acetylcysteine (NAC) formulation administered in accordance with these methods comprises a solid core composition comprising about 133 mg of rifaximin and about 561.3 mg of NAC.

In various embodiments, the rifaximin and N-acetylcysteine (NAC) formulation administered in accordance with these methods comprises a solid core composition comprising about 59-73 mg or 62-70 mg of rifaximin and about 405-616 mg or 427-588 mg or 450-560 mg or 504-616 mg or 532-588 mg of NAC.

In various embodiments, the rifaximin and N-acetylcysteine (NAC) formulation administered in accordance with these methods comprises a solid core composition comprising about 118-146 mg or 125-139 mg of rifaximin and about 405-616 mg or 427-588 mg or 450-560 mg or 504-616 mg or 532-588 mg of NAC.

In various embodiments, the solid core composition in the rifaximin and N-acetylcysteine (NAC) formulation further comprises one or more excipients.

In various embodiments, the solid core composition in the rifaximin and N-acetylcysteine (NAC) formulation is in a tablet form.

In various embodiments, the coating around the solid core composition comprises a polymer.

In various embodiments, the coating around the solid core composition comprises at least one polymer that is insoluble at a pH lower than 5. In various embodiments, the coating of the second solid composition comprises at least one polymer that is insoluble at a pH lower than 3.5. In various embodiments, the coating of the second solid composition comprises at least one polymer that is insoluble at a pH between 1.5-3.5.

In various embodiments, the coating around the solid core composition does not comprise gelatin. In various embodiments, the coating around the solid core composition comprises hypromellose.

In various embodiments, the solid core composition comprising the rifaximin and NAC is substantially undissolved after 1 hour in a Type 2 dissolution bath containing about 0.1N hydrochloric acid.

In various embodiments, the solid core composition comprising the rifaximin and NAC entirely disperses or substantially disburses in fed state simulated intestinal fluid.

In various embodiments of these methods a rifaximin and N-acetylcysteine (NAC) formulation is orally administered to the subject in need thereof, the formulation comprises: a solid core composition comprising an amount of rifaximin and an amount of NAC, and a coating around the solid core composition, wherein the solid core composition comprises a single layer that is uniform or substantially uniform in composition.

The rifaximin provided in these formulations can be rifaximin base, rifaximin polymorphs (e.g., rifaximin-α), amorphous rifaximin, pharmaceutically acceptable salts thereof, solvates thereof, hydrates thereof, or enantiomers thereof, unless otherwise noted. In particular embodiments, the rifaximin provided in these formulations is rifaximin base. In other particular embodiments, the rifaximin provide in these formulations is rifaximin polymorph-α. In other particular embodiments, the rifaximin provide in these formulations is the amorphous form of rifaximin.

As nonlimiting examples, the rifaximin is at least 95%, 96%, 97%, 98%, 99%, 99.5%, 99.6%, 99.7%, 99.8%, or 99.9% in the form that is provided. For example, if rifaximin is provided in the rifaximin-α form, at least 95%, 96%, 97%, 98%, 99%, 99.5%, 99.6%, 99.7%, 99.8%, or 99.9% of the rifaximin will be in the rifaximin-α form. If rifaximin is provided in as rifaximin base, at least 95%, 96%, 97%, 98%, 99%, 99.5%, 99.6%, 99.7%, 99.8%, or 99.9% of the rifaximin will be in the rifaximin base form. In various embodiments, rifaximin base form is not purified for polymorphs.

In various embodiments, the amount of rifaximin in the rifaximin and NAC formulation administered is not 200 mg. In various embodiments, the amount of NAC in the rifaximin and NAC formulation is not 600 mg of NAC. In various embodiments, the amount of rifaximin and the amount of NAC in the rifaximin and NAC formulation administered is not 200 mg of rifaximin and 600 mg of NAC.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

In vitro dilution growth/cidal experiments were conducted on E. Coli and Klebsiella to determine doses of rifaximin and NAC, including lowest doses of rifaximin and lowest doses of NAC. Based on the experiments, NAC concentration of 0.125 mg/mL showed improve the minimal inhibitory concentration (MIC) for rifaximin at 4 μg/mL.

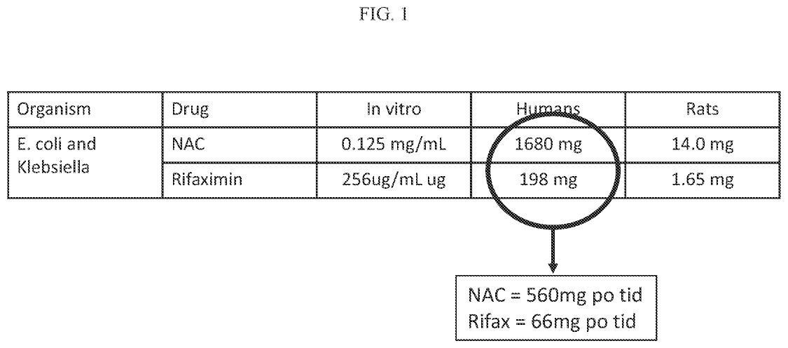

Dosing conversion for use in a rat model and use in humans were calculated and is shown in FIG. 1. A NAC dose of 560 p.o. three times a day along with a rifaximin dosage of 66 mg p.o. three times a day provided for a total dosage of 1680 mg NAC and 198 mg rifaximin per day in humans.

To calculate the human dose for NAC and Rifaximin, FDA Guidance for Industry (Estimating the Maximum Safe Starting Dose in Initial Clinical Trials for Therapeutics in Adult Healthy Volunteers—www.fda.gov/media/72309/download) was used, where the conversion of animal dose to human equivalent doses was based on body surface area (Table 1 from the guideline). The NAC rat dose of approximately 58 mg/kg (14 mg of NAC for each rat with an average weight of 0.24 kg) was divided by the conversion factor of 6.2 and multiplied by 60 kg (the average weight of an adult human, as recommended by the FDA), resulting in 561.3 mg per dose. The rifaximin rat dose of approximately 6.87 mg/kg (1.65 mg of rifaximin for each rat with an average weight of 0.24 kg) was divided by the conversion factor of 6.2 and multiplied by 60 kg (the average weight of an adult human, as recommended by the FDA), resulting in 66.5 mg per dose.

### Example 2

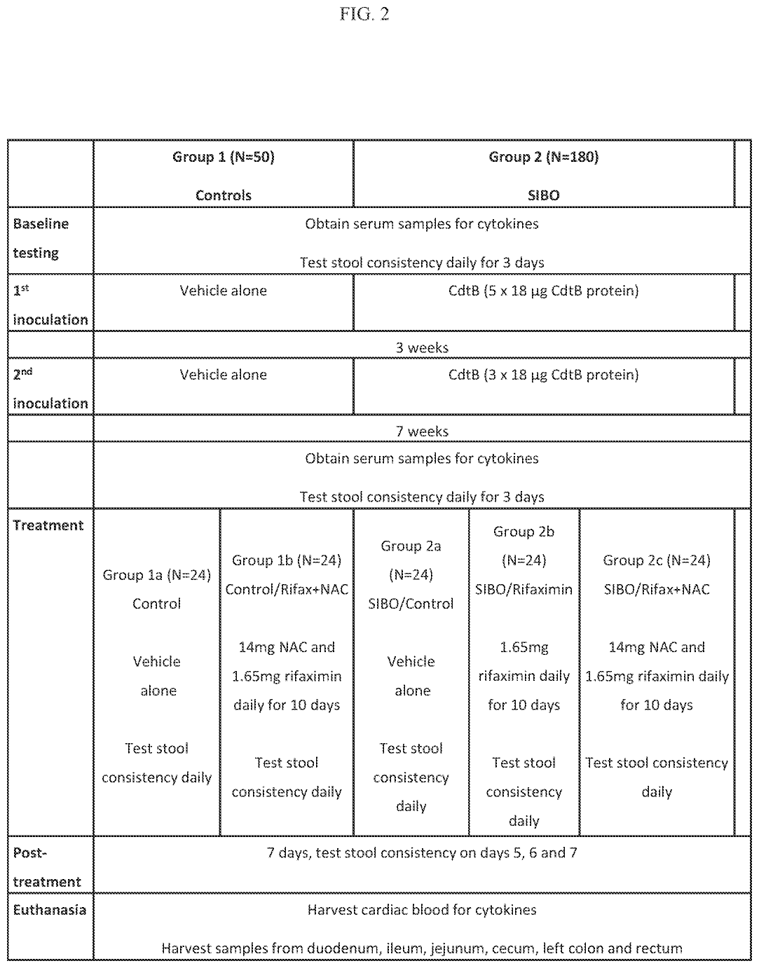

A validated rat model of IBS-D was used for experiments to test the dosages for NAC and rifaximin. The study plan is shown in FIG. 2. Rats were gavaged with the control vehicle (1 ml of 1× PBS), rifaximin, or rifaximin+NAC as indicated in FIG. 2.

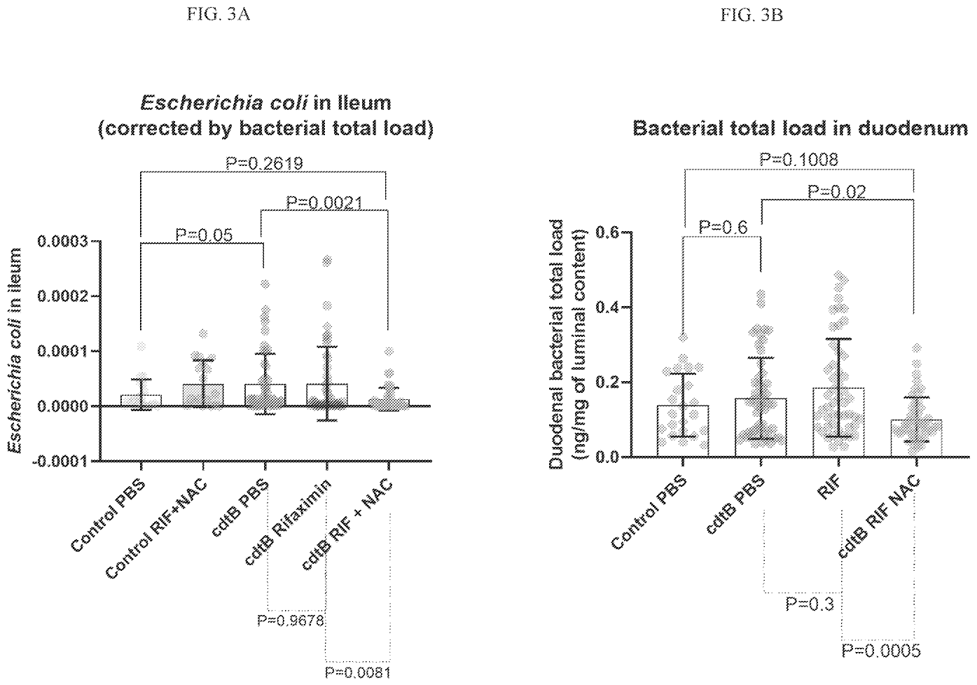

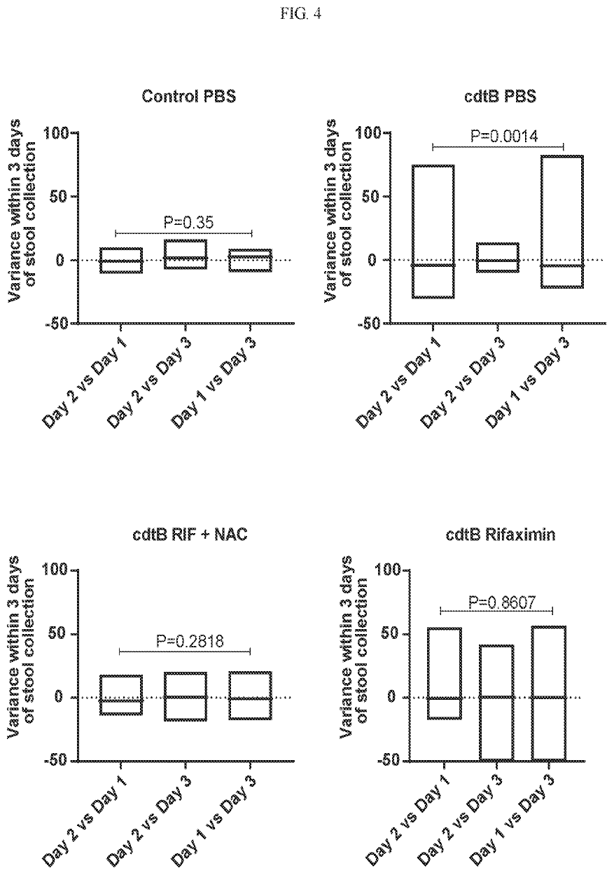

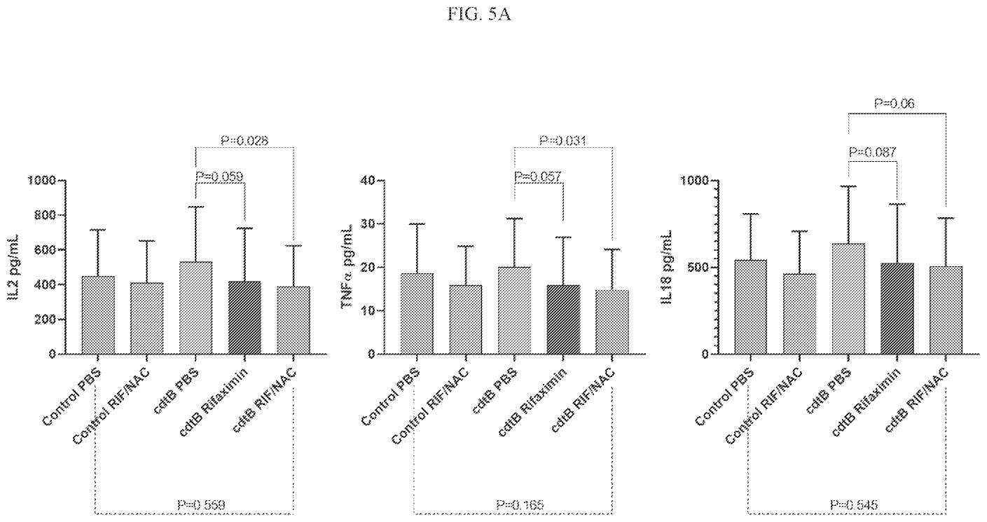

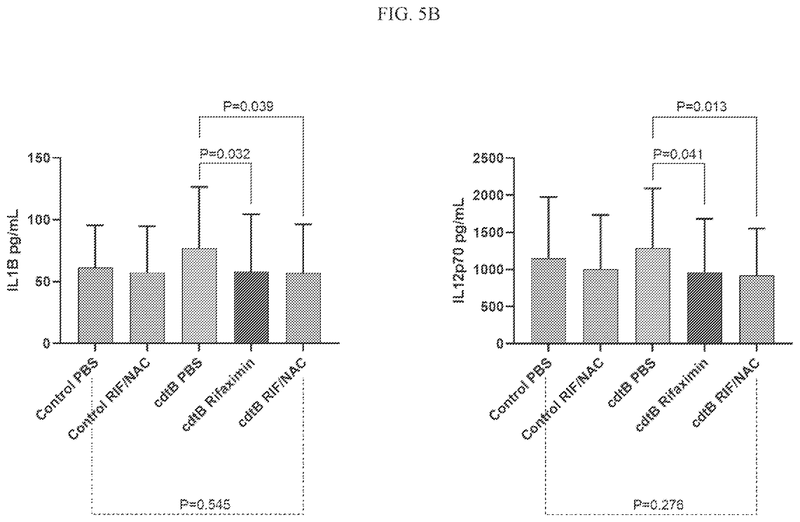

The results show therapy with Rifaximin+NAC decreases Escherichia coli load and total bacterial load in the small bowel (FIG. 3A and FIG. 3B); therapy with Rifaximin+NAC improves stool consistency (FIG. 4); therapy with Rifaximin+NAC decreases systemic inflammation (FIG. 5A and FIG. 5B).

In conclusion, NAC improves the effectiveness of rifaximin in killing E. coli and Klebsiella. This effect is so potent that it allows a much reduced dose of rifaximin with higher effectiveness. An effective dose of combination in humans estimated to be rifaximin 66 mg+NAC 560 mg, both TID.

Rifaximin with NAC is superior to rifaximin alone in treating IBS and reducing small bowel E. coli (Sprague-Dawley rats do not have Klebsiella). Rats treated with combination had a greater reduction in stool wet weight.

Cytokine profiles predict the response seen observed in this study.

### Example 3

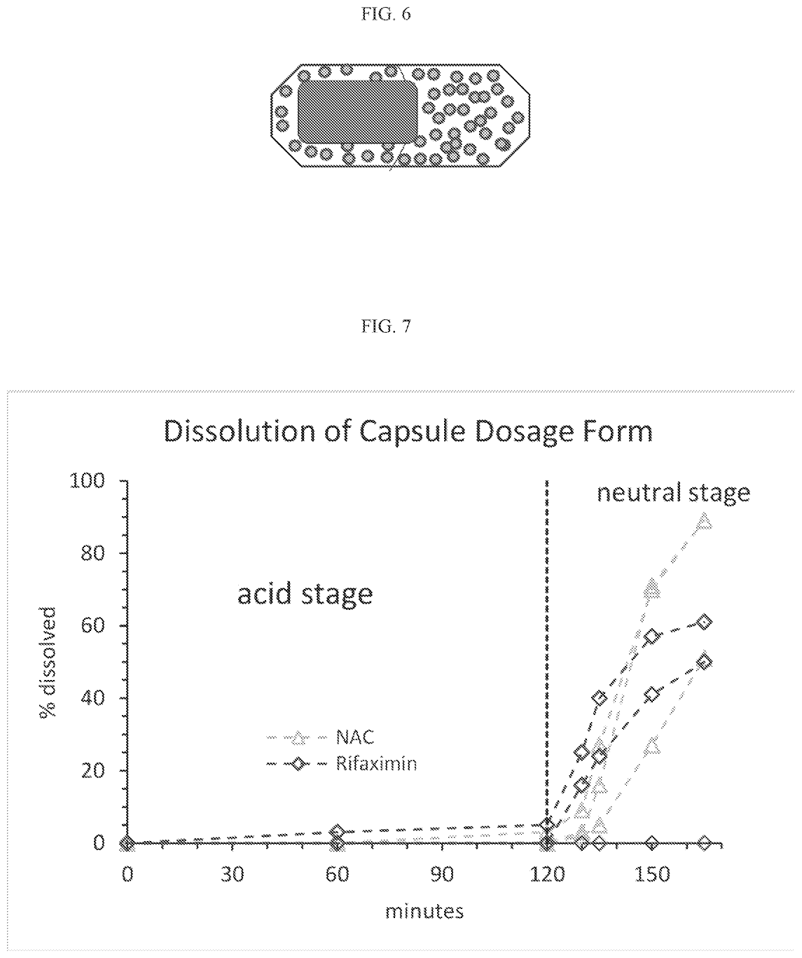

FIG. 6 depicts an exemplary embodiment of a rifaximin and NAC formation described herein. FIG. 6 shows a capsule containing a solid tablet comprising rifaximin, and minitablets comprising NAC.

FIG. 7 shows the dissolution percentage of rifaximin and NAC over a period of time of 0-175 minutes, and the change from an acidic solution of pH 1 to a neutral solution.

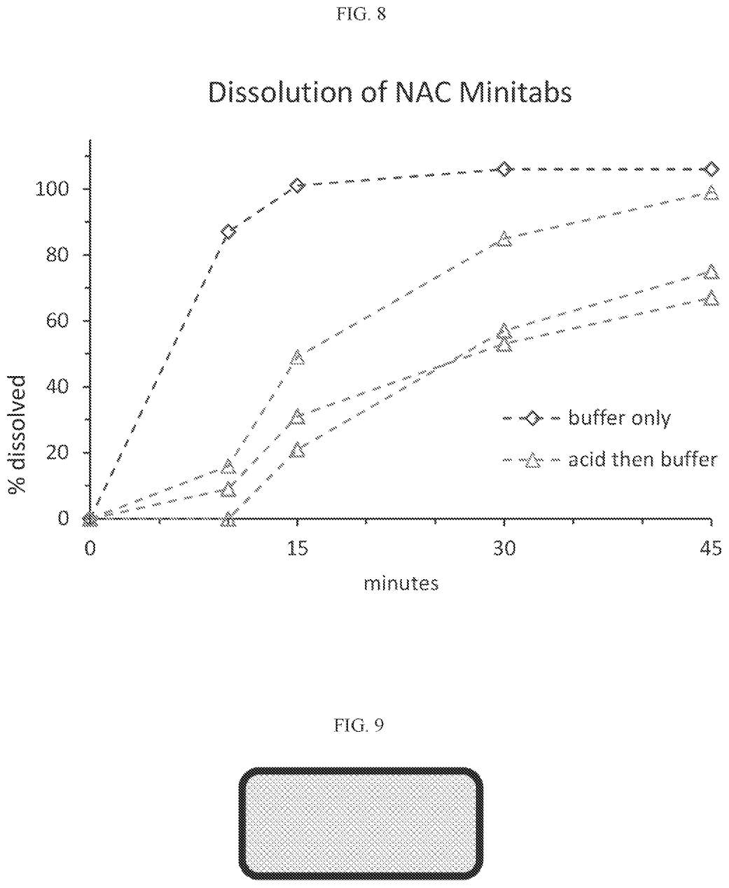

FIG. 8 shows the dissolution of the NAC minitablets over a period of time of 0-45 minutes in buffer only (100 mM sodium phosphate pH 7.4 with 0.45% sodium lauryl sulfate), and in acidic solution (pH 1 (0.1N HCl) with no surfactant, followed by the buffer. Percent dissolution is based on the labeled amount of the drug in the dosage form. In this instant example, due to variability from unit to unit it may result in some dosage form units containing more than the target dose, which results in dissolution values to appear over 100%.

### Example 4

FIG. 9 depicts an exemplary embodiment of a rifaximin and NAC formation described herein. FIG. 9 shows a solid core composition comprising rifaximin and NAC, surrounded by a coating.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the invention described herein have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the invention described herein, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A rifaximin and N-acetylcysteine (NAC) formulation, comprising:
a shell;
a first solid composition comprising an amount of rifaximin within the shell; and
a plurality of a coated second solid composition comprising an amount of NAC within the shell.

2. A rifaximin and N-acetylcysteine (NAC) formulation, comprising:
a solid core composition comprising an amount of rifaximin and an amount of NAC; and
a coating around the solid core composition.

3. The formulation of claim 1, wherein the amount of rifaximin is about 66 mg or about 132 mg.

4. (canceled)

5. The formulation of claim 1, wherein the amount of rifaximin is about 59-73 mg or about 118-146 mg.

6. (canceled)

7. (canceled)

8. (canceled)

9. The formulation of claim 1, wherein the amount of NAC is about 560 mg.

10. (canceled)

11. (canceled)

12. (canceled)

13. (canceled)

14. The formulation of claim 1, wherein the amount of NAC is about 405-616 mg.

15. The formulation of claim 1,
wherein the first solid composition further comprises one or more excipients, or
wherein the first solid composition is in a tablet form.

16. The formulation of claim 1,
wherein the second solid composition further comprises one or more excipients, or
wherein the second solid composition is in form of a mini-tablet, crystal, a pellet, or a bead, or
wherein the coating of the second solid composition comprises at least one polymer, or
wherein the coating of the second solid composition comprises at least one polymer that is insoluble at a pH lower than 5.

17. (canceled)

18. (canceled)

19. (canceled)

20. (canceled)

21. The formulation of claim 1, wherein the coating of the second solid composition comprises at least one polymer that is insoluble at a pH lower than 3.5.

22. The formulation of claim 1, wherein the coating of the second solid composition comprises at least one polymer that is insoluble at a pH between 1.5-3.5.

23. The formulation of claim 1,
wherein the shell does not comprise gelatin, or
wherein the shell comprises hypromellose.

24. (canceled)

25. The formulation of claim 1,
wherein first solid composition comprising the rifaximin is substantially undissolved after 1 hour in a Type 2 dissolution bath containing about 0.1N hydrochloric acid, or
wherein the first solid composition comprising the rifaximin substantially disperses in fed state simulated intestinal fluid.

26. (canceled)

27. (canceled)

28. (canceled)

29. The formulation of claim 1,
wherein the first solid composition comprising the rifaximin is a tablet comprising rifaximin and one or more excipients;
wherein the plurality of coated second solid composition comprising NAC is a plurality of a mini-tablet comprising NAC and one or more excipients; and
wherein the coating comprises at least one polymer that is substantially insoluble at a pH lower than 3.5.

30. A method of treating irritable bowel syndrome (IBS), comprising:
administering one or more doses of a formulation of claim 1, to a subject in need thereof.

31. The method of claim 30, wherein the IBS is diarrhea predominant IBS (IBS-D), or wherein the IBS is hydrogen sulfide (H₂S) positive IBS.

32. (canceled)

33. A method of treating small intestinal bacterial overgrowth (SIBO), comprising: administering one or more doses of a formulation of claim 1, to a subject in need thereof.

34. A method of decreasing Escherichia coli (E. Coli) load, Klebsiella load, and total bacterial load in the small bowel, comprising: administering one or more doses of a formulation of claim 1, to a subject in need thereof.

35. The method of claim 34, wherein the decrease in the E. Coli load, Klebsiella load, and the total bacterial load occurs in the ileum or duodenum.

36. (canceled)

37. A method of improving stool consistency, reducing systemic inflammation, or both, comprising: administering one or more doses of a formulation of claim 1, to a subject in need thereof.

38. The method of claim 33, wherein administering one or more doses of the formulation comprises administering three dosages per day to the subject in need thereof, or wherein administering one or more doses of the formulation comprises administering three dosages per day for about 7 to 10 days to the subject in need thereof.

39. (canceled)

40. (canceled)

41. The formulation of claim 2, wherein the amount of rifaximin is about 66 mg or about 132 mg.

42. The formulation of claim 2, wherein the amount of rifaximin is about 59-73 mg or about 118-146 mg.

43. The formulation of claim 2, wherein the amount of NAC is about 560 mg.

44. The formulation of claim 2, wherein the NAC in the solid core composition increases the dissolution of rifaximin relative to a solid core composition without NAC.
